(19) United States
(12) Patent Application Publication
(10) Pub. No.: US 2005/0070594 A1
(43) Pub. Date: Mar. 31, 2005

(54) Use of angiotensin II receptor antagonists

(75) Inventors: Stefan Kauschke, Biberach (DE); Michael Mark, Biberach (DE); Ulrich Kintscher, Berlin (DE); Michael Schupp, Berlin (DE); Thomas Unger, Berlin (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 10/899,784

(22) Filed: Jul. 27, 2004

Related U.S. Application Data
(60) Provisional application No. 60/503,317, filed on Sep. 16, 2003.
(60) Provisional application No. 60/514,998, filed on Oct. 28, 2003.
(60) Provisional application No. 60/534,516, filed on Jan. 6, 2004.

(30) Foreign Application Priority Data
Jul. 31, 2003 (DE) DE 103 35 027
Oct. 6, 2003 (DE) DE 103 46 260
Dec. 5, 2003 (DE) DE 103 56 815

(51) Int. Cl. A61K 31/401

(52) U.S. Cl. 514/423

(57) ABSTRACT

The invention relates to the use of angiotensin II receptor antagonists for treating people in whom type 2 diabetes mellitus has been diagnosed or who are suspected of prediabetes, for preventing diabetes or for treating metabolic syndrome and insulin resistance in patients with normal blood pressure.

### APPLICATION DATA

This application claims benefit to DE 103 35 027 filed Jul. 31, 2003, DE 103 46 260 filed Oct. 6, 2003, DE 103 56 815 filed Dec. 5, 2003 and U.S. provisional application No. 60/503,317 filed Sep. 16, 2003, Ser. No. 60/514,998 filed Oct. 28, 2003 and Ser. No. 60/534,516 filed Jan. 6, 2004.

### FIELD OF THE INVENTION

The invention relates to the field of the angiotensin II receptor antagonists and relates to their use for treating people in whom diabetes has been diagnosed or who are suspected of prediabetes, for preventing diabetes or for treating metabolic syndrome and insulin resistance in patients with normal blood pressure.

### BACKGROUND OF THE INVENTION

Type 2 diabetes mellitus is the manifestation of two pathophysiological phenomena, namely a reduced secretion of insulin from the beta cells of the pancreas and insulin resistance in the target organs of the liver, skeletal musculature and fatty tissue. As a rule there is a complex disruption of both components. The disease is diagnosed as fasting hyperglycaemia, i.e. the blood sugar concentration after 10-12 hours' fasting is above the threshold of 125 mg of glucose per dl of plasma. Controlled treatment of manifest type 2 diabetes can be achieved using compounds of the category of the thiazolidinediones (glitazones). These compounds improve the utilisation of circulating insulin and thus result in a lowering of the blood sugar levels (insulin sensitisers). At the same time the increased insulin levels are reduced by feedback mechanisms and in this way the load on the pancreas is relieved. Insulin sensitisers such as troglitazone, rosiglitazone or pioglitazone develop this activity by binding to specific nuclear receptors known as PPAR-gamma (Peroxisomal Proliferator Activated Receptor). These act as transcription regulators for a number of genes which are important to glucose and lipid metabolism. By means of this function, PPAR-gamma ligands such as prostaglandins or the synthetic thiazolidinediones (glitazones) may contribute to the treatment of type 2 diabetes. One of the main mechanisms for lowering glucose by PPAR-gamma ligands is the induction of the differentiation of adipocytes. Increased adipocyte differentiation and remodelling of the fatty tissue brought about by PPAR-gamma ligands leads to a diversion or redistribution of free fatty acids from the skeletal muscle into the fatty tissue, thereby increasing the glucose metabolism in the muscles.

As every second type 2 diabetes patient show signs of coronary heart disease at the time of diagnosis, for example, the causes of diabetes are increasingly suspected to reside in a complex metabolic disorder which may be indicated by a number of risk factors such as abnormal glucose tolerance, increased fasting blood sugar, insulin resistance, high blood pressure, dyslipidaemia or centripetal obesity. The prevalence of insulin resistance is particularly marked in patients with hypertriglyceridaemia and low HDL-cholesterol. Reference is made to pre-type 2 diabetes, metabolic syndrome, syndrome X or insulin resistance syndrome. In a first phase a reduced insulin response by the target organs causes an increase in the pancreatic insulin secretion in order to keep the blood sugar level in the normal range. After a number of years of excessive or increasing insulin production there comes a time when the insulin secretion by the beta cells of the pancreas cannot be increased any further. The phase of abnormal glucose tolerance then begins. The body can no longer absorb glucose peak values fast enough. Finally, if the fasting blood sugar remains persistently high, diabetes is manifest.

WO 95/06410 discloses the use of angiotensin II receptor antagonists for treating chronic inflammatory diseases including systemic autoimmune diseases. Diabetes is mentioned as one of a number of examples of systemic autoimmune diseases. The autoimmune diseases include type 1 diabetes mellitus which occurs mainly in young people under 30 years of age with a genetic predisposition, in whom insulitis occurs under the influence of various factors with subsequent destruction of the B cells so that the pancreas can only produce a little insulin or none at all. Type 2 diabetes mellitus is not regarded as an autoimmune disease.

Angiotensin II receptor antagonists are used to treat high blood pressure and consequent injury to cardiovascular organs which are brought into contact with high blood pressure. In the specialist literature they are generally categorised as metabolically neutral. Improvement to the insulin sensitivity in the animal model brought about by the active substance irbesartan is reported by Henriksen et al (Hypertension 38:884-90, 2001).

The aim of the present invention is to provide a pharmaceutical composition which can be used both to treat manifest type 2 diabetes and to treat the first signs of the complex metabolic disorder of prediabetes and thereby prevent type 2 diabetes mellitus. Within the scope of the present invention it has now surprisingly been found that a few angiotensin II receptor antagonists and their salts not only act to reduce blood pressure, in known manner, but are also capable of increasing the expression of genes in a cellular system, the transcription of which is known to be regulated by the PPARgamma receptor. This opens up new therapeutic possibilities in the treatment and prevention of type 2 diabetes, metabolic syndrome and insulin resistance. In order to ensure comparable conditions this effect is observed and quantified within the scope of the present invention by means of a stably transformed cell line (cf. Example 2). The cells used are CHO cells which are the result of transformation with two gene constructs. The first of these constructs codes for the luciferase gene from Photinus pyralis (de Wet J R, Mol Cell Biol (1987) 7:725) under the control of a synthetic promoter with a five-fold repeat of a yeast Gal4-binding site (cf. GeneBank Sequence AF058756). The second construct codes for a fusion protein consisting of the ligand binding domain of the human PPARgamma2 transcription factor (cf. GeneBank Sequence U79012) and the yeast GAL4 DNA binding domain (Amino acids 1-147; Sadowski I, Nucleic Acids Res (1989) 17:7539).

The induction of the transcription of PPARgamma-regulated genes is known from the thiazolidinediones used as antidiabetic drugs (e.g. rosiglitazone) and is brought about by their binding to the PPARgamma Receptor and its activation. Within the scope of the test system used here this effect may be quantified as an induced luciferase activity of the transformed cell line. The same induction of a luciferase activity takes place with the angiotensin II receptor antagonists, contrary to expectation, not by the binding of the active substance to the PPARgamma Receptor. The induction is particularly marked for the active substance telmisartan. Binding of e.g. telmisartan to the PPARgamma receptor cannot be detected in various test systems. It is therefore presumed that the increase in the affinity of cofactor proteins for PPARgamma caused by an angiotensin II receptor antagonist such as telmisartan also leads to the recruiting of the cofactor proteins if there are no high-affinity synthetic PPARgamma ligands present. This then brings about activation of the transcription of genes regulated by the PPARgamma receptor, this activation being mediated by these cofactors. As the induction of these genes is responsible for the anti-diabetic activity of the thiazolidinediones it can be assumed that the induction of the same genes by angiotensin II receptor antagonists such as telmisartan results in a comparable anti-diabetic activity. Thus, these active substances are suitable not only for treating high blood pressure but also for treating and preventing type 2 diabetes mellitus.

The discovery of this new therapeutic effect of angiotensin II receptor antagonists and the salts thereof means that they can be used to produce a pharmaceutical composition for the treatment of people in whom type 2 diabetes mellitus has been diagnosed or who are suspected of prediabetes, for preventing diabetes or for treating metabolic syndrome and insulin resistance in patients with normal blood pressure. They are particularly suitable for the treatment and prevention of type 2 diabetes and pre-type 2 diabetes. This includes the treatment and prevention of metabolic syndrome, syndrome X or insulin-resistance syndrome. When this invention refers to persons requiring treatment, it relates primarily to treatment and prevention in humans, but the active substances and combinations of active substances used may also be used accordingly in veterinary medicine on mammals.

Type 2 diabetes mellitus manifests itself in a fasting blood sugar level exceeding 125 mg of glucose per dl of plasma; the measurement of blood glucose values is a standard procedure in routine medical analysis. If a glucose tolerance test is carried out, the blood sugar level of a diabetic will be in excess of 200 mg of glucose per dl of plasma 2 hours after 75 g of glucose have been taken on an empty stomach. In a glucose tolerance test 75 g of glucose are administered orally to the patient being tested after 10-12 hours of fasting and the blood sugar level is recorded immediately before taking the glucose and 1 and 2 hours after taking it. In a healthy subject the blood sugar level before taking the glucose will be between 60 and 110 mg per dl of plasma, less than 200 mg per dl 1 hour after taking the glucose and less than 140 mg per dl after 2 hours. If after 2 hours the value is between 140 and 200 mg this is regarded as abnormal glucose tolerance.

If insulin resistance can be detected this is a particularly strong indication of the presence of prediabetes. Thus, it may be that in order to maintain glucose homoeostasis a person needs 2-3 times as much insulin as another person, without this having any direct pathological significance. The most certain method of determining insulin resistance is the euglycaemic-hyperinsulinaemic clamp test. The ratio of insulin to glucose is determined within the scope of a combined insulin-glucose infusion technique. There is found to be insulin resistance if the glucose absorption is below the 25th percentile of the background population investigated (WHO definition). Rather less laborious than the clamp test are so called minimal models in which, during an intravenous glucose tolerance test, the insulin and glucose concentrations in the blood are measured at fixed time intervals and from these the insulin resistance is calculated. Another method of measurement is the mathematical HOMA model. The insulin resistance is calculated by means of the fasting plasma-glucose and the fasting insulin concentration. In this method it is not possible to distinguish between hepatic and peripheral insulin resistance. These processes are not really suitable for evaluating insulin resistance in daily practice. As a rule, other parameters are used in everyday clinical practice to assess insulin resistance. Preferably, the patient's triglyceride concentration is used, for example, as increased triglyceride levels correlate significantly with the presence of insulin resistance.

To simply somewhat, in practice it is assumed that people are insulin-resistant if they have at least 2 of the following characteristics:
1) overweight or obesity
2) high blood pressure
3) dyslipidaemia (an altered content of total lipids in the blood)
4) at least one close relative in whom abnormal glucose tolerance or type 2 diabetes has been diagnosed.

Overweight means in this instance that the Body Mass Index (BMI) is between 25 and 30 kg/m², the BMI being the quotient of the body weight in kg and the square of the height in metres. For obesity the BMI is more than 30 kg/m². It is immediately apparent, from the above definition of insulin resistance, that hypotensive agents are suitable and indicated for treating it if, among other things, high blood pressure is found in the patient. One result of the present invention is that some angiotensin II receptor blockers, but particularly telmisartan, are preferred hypotensives by virtue of their property of PPAR-gamma activation, and are suitable for treating insulin resistance even when the patient's blood pressure is not high but normal. Thus, type 2 diabetics can be treated with telmisartan at the same time as receiving a primary or back-up treatment for dyslipidaemia. Conventional dosages of telmisartan significantly reduce the plasma levels of LDL-cholesterol, total cholesterol and/or triglycerides.

As insulin resistance is regarded as a condition which brings about a gradual increase in blood pressure, treatment with telmisartan in spite of normal blood pressure levels can be regarded as high blood pressure prevention.

A similar indication of prediabetes is if the conditions for metabolic syndrome are met, the main feature of which is insulin resistance. According to the ATP IHINCEP Guidelines (Executive Summary of the Third Report of the National Cholesterol Education Program (NCEP) in the Journal of the American Medical Association 285:2486-2497, 2001) metabolic syndrome is present if a patient has at least 3 of the following characteristics:
1) Abdominal obesity, defined as a waist measurement of >40 inches or 102 cm in men and >35 inches or 94 cm in women
2) Triglyceride levels >150 mg/dl
3) HDL-cholesterol levels <40 mg/dl in men
4) High blood pressure >130/>85 mm Hg
5) Fasting blood sugar of >110 mg/dl

This definition of metabolic syndrome immediately shows that hypotensives are suitable for treating it if the patient is found to have high blood pressure, among other things. One result of the present invention is that some angiotensin II receptor blockers, but especially telmisartan, are preferred hypotensives on account of the property of PPAR-gamma activation, and are suitable for treating insulin resistance even if the patient is not found to have high blood pressure. As metabolic syndrome is also regarded as a condition which causes a gradual rise in blood pressure, its treatment with telmisartan can also be regarded as prevention of high blood pressure, in spite of normal blood pressure levels.

There is also a suspicion of prediabetes if the fasting blood sugar level is above the normal maximum level of 110 mg of glucose per dl of plasma but does not exceed the threshold of 125 mg of glucose per dl of plasma which indicates diabetes. Another indication of prediabetes is abnormal glucose tolerance, i.e. a blood sugar level of 140-200mg of glucose per dl of plasma 2 hours after taking 75 g of glucose after a fast within the scope of a glucose tolerance test.

A triglyceride blood level of more than 150 mg/dl also indicates the presence of pre-diabetes. This suspicion is confirmed by a low blood level for HDL cholesterol. In women, levels below 40 mg per dl of plasma are regarded as too low while in men levels below 50 mg per dl of plasma are regarded as too low. Triglycerides and HDL cholesterol in the blood can also be determined by standard methods in medical analysis and are described for example in Thomas L (Editor): “Labor und Diagnose”, TH-Books Verlagsgesellschaft mbH, Frankfurt/Main, 2000. A suspicion of prediabetes is further confirmed if the fasting blood sugar levels also exceed 110 mg of glucose per dl of plasma. If the blood levels measured are in the region of these threshold values, the ratio of the waist measurement to the hip measurement can be used as an additional aid to make the decision. If this ratio exceeds a value of 0.8 in women or 1 in men, treatment is indicated.

Angiotensin II receptor antagonists are particularly indicated for treating diabetes or suspected prediabetes if hypertension also has to be treated. This is the case if the systolic blood pressure exceeds a value of 140 mm Hg and diastolic blood pressure exceeds a value of 90 mm Hg. If a patient is suffering from manifest diabetes it is currently recommended that the systolic blood pressure be reduced to a level below 130 mm Hg and the diastolic blood pressure be lowered to below 80 mm Hg. To achieve these levels it may be indicated in certain cases to combine angiotensin II receptor antagonists with a diuretic or a calcium antagonist. The term “diuretic” included thiazides or thiazide analogues such as hydrochlorothiazides (HCTZ), clopamide, xipamide or chlorthalidone, aldosterone antagonists such as spironolactone or eplerenone and also other diuretics suitable for treating high blood pressure such as furosemide and piretanide, and combinations thereof with amiloride and triamterene.

The present invention means that for subjects being treated for increased blood pressure, angiotensin II receptor antagonists such as telmisartan are indicated whenever the development of diabetes is to be prevented or manifest diabetes is to be treated.

In only 10% of all cases of elevated blood pressure (secondary hypertension) is it possible to determine an identifiable course such as e.g. kidney disease. As a rule, secondary hypertension can be remedied by treating and removing the cause. However, in almost 90% of all cases it is primary hypertension, the exact cause of which is not known and which therefore cannot be directly cured. The negative effects of elevated blood pressure can be reduced by changing lifestyle and correct treatment. The interaction of different risk factors or the combined occurrence of individual risk factors appear to cause high blood pressure. In particular, the combination of high blood pressure with disorders of the fat and sugar metabolism is observed to an increasing extent. These disorders are often unnoticed to begin with but can be recognised from increased blood levels of triglycerides and glucose and lower blood levels of HDL cholesterol. At a fairly advanced stage they can also be detected in slowly increasing corpulence. These disorders can be explained by increasing insulin resistance. The less effective the insulin, the more the fat and sugar metabolisms are disrupted. The combination of all these disorders in the last analysis increases the probability of contracting the sugar disease diabetes and dying prematurely of heart or vascular disease.

Estimates are based on the supposition that about a third of adults in those parts of the world with an excessive supply of food are affected by the combination of high blood pressure and disorders of the fat and sugar metabolism and that this number will continue to increase. Consequently there is a need for drugs which are capable of helping to slow down or stop the progress of the above-mentioned metabolic disorders at the earliest possible stage and at the same time to obviate the detrimental effects of increased blood pressure on the health.

The present invention also discloses a pharmaceutical composition which can be used both to treat hypertension and to treat manifest type 2 diabetes or the first signs of the complex metabolic disorder of prediabetes. Thus, the invention also includes diabetes prevention in patients who are being treated for high blood pressure. If therefore a suitable angiotensin II receptor antagonist such as telmisartan is used immediately to control blood pressure as soon as one of the above-mentioned signs of prediabetes is present, the onset of manifest type 2 diabetes can be delayed or prevented.

Angiotensin II receptor antagonists which are suitable within the scope of the present invention are compounds for which binding to the PPARgamma ligand binding domain can be ruled out by in vitro tests (cf. Example 1), while they activate the expression of a stably transfected luciferase gene at cellular level, i.e. after the addition of a stably transformed PPARgamma reporter cell line to the culture medium (cf. Example 3).

Suitable angiotensin II receptor antagonists also exhibit
no in vitro binding to the ligand binding domain of a human PPARgamma receptor, but lead to the
induction of a luciferase activity when they are added to the culture medium of a stably transformed PPARgamma reporter cell line which
   a) expresses a fusion protein consisting of the ligand binding domain of the human PPARgamma transcription factor and the yeast GAL4 DNA binding domain and
   b) a luciferase gene under the control of a five-times repeated yeast Gal4 binding site.

The preparation of a PPARgamma reporter cell line of this kind is described in Example 2.

There is no in vitro binding to the ligand binding domain of the human PPARgamma2 receptor if it cannot be detected in an AlphaScreen (Ullmann E F et al, Proc Natl Acad Sci USA (1994) 91:5426-5430). Instead of an Alpha Screen, an SPA assay (Mukherjee R et al., J Steroid Biochem Mol Biol (2002) 81:217-225) or an NMR investigation (Johnson B A et al., J Mol Biol (2000) 298:187-194) may also be carried out. As a rule, binding to the receptor cannot be detected by any of these methods.

A comprehensive list of angiotensin II receptor antagonists can be found on pages 7-18 of WO 95/26188. Angiotensin II receptor antagonists are described inter alia in EP-A-253310, EP-A-323841, EP-A-324377, EP-A-420237, EP-A-443983, EP-A-459136, EP-A-475206, EP-A-502314, EP-A-504888, EP-A-514198, WO 91/14679, WO 93/20816, US 4,355,040 and US 4,880,804. Forms which are frequently mentioned are sartans, such as candesartan, eprosartan, irbesartan, losartan, olmesartan, tasosartan, telmisartan or valsartan. Those which are particularly preferred according to the present invention are irbesartan, losartan und telmisartan. The best results are clearly obtained with telmisartan and the salts thereof. The formulations produced contain an equivalent of 20-200 mg, preferably 20, 40, 80, 120, 160 or 200 mg of the free acid of the active substance. If the active substance is combined with HCTZ or chlorthalidone, the formulation contains 10-50 mg, preferably 50, 25 or 12.5 mg of the diuretic.

The advantageous activity of individual angiotensin II antagonists disclosed within the scope of this invention is particularly marked for the active substance telmisartan. If it appears useful or necessary to use an angiotensin II receptor blocker in conjunction with one or more other therapeutic active substances, telmisartan is a preferred angiotensin II receptor blocker, as it combines a blood pressure lowering and metabolic activity in a single active substance, e.g. an antidiabetic activity which also helps to prevent diabetes. For this reason, preformulated active substance combinations of telmisartan with HMG-Co A reductase inhibitors such as simvastatin or atorvastatin constitute a major further development in the treatment of cardiovascular, cardiopulmonary, pulmonary or renal diseases, but also in the treatment of dyslipidaemia, osteoporosis or Alzheimers. This also applies to active substance combinations of telmisartan with rosiglitazone or pioglitazone or repaglinide or mefformin or a DPP4 inhibitor in the treatment of diabetes. Telmisartan must also be regarded as a preferred RAS inhibitor in the treatment of high blood pressure with inhibitors of the renin-angiotensin system (RAS) combined with a calcium antagonist such as amlodipine or nifedipine or an aldosterone antagonist such as spironolactone or eplerenone. The combination with an aldosterone antagonist such as eplerenone also represents an important development in the treatment or prevention of weak heart or heart attack.

In addition to raised blood pressure, lipid metabolism disorders (dyslipidaemias) and diabetes mellitus also mean an increased risk of stroke, with the result that telmisartan, also in conjunction with thrombocyte aggregation inhibitors such as clopidogrel or dipyridamole and additionally combined with acetylsalicylic acid (ASA), also constitutes a preferred combination partner, particularly for preventing strokes. For this purpose dipyridamole can be used in a dosage from 50 to 750 mg, preferably from 100 to 500 mg and particularly from 200 to 450 mg. ASA may be used in a dosage from 10 to 200 mg, preferably from 25 to 100 mg and particularly from 30 to 75 mg.

Therefore the present invention further relates to pharmaceutical compositions containing telmisartan or one of the salts thereof combined with
amlodipine or nifedipine,
eplerenone or spironolactone,
simvastatin or atorvastatin,
rosiglitazone or pioglitazone or repaglinide or mefformin,
dipyridamole or clopidogrel, optionally combined with acetylsalicylic acid,
a sulphonylurea,
an aldosterone antagonist,
an HMG-Co A reductase inhibitor,
a DPP4 inhibitor or
a thrombocyte aggregation inhibitor,
   and the preparation thereof. These preformulated combinations of active substances are generally incorporated with one or more formulation adjuvants such as mannitol, sorbitol, xylitol, saccharose, calcium carbonate, calcium phosphate, lactose, croscarmellose sodium salt (cellulose carboxymethylether sodium salt, cross-linked), crospovidone, sodium starch glycolate, hydroxypropylcellulose (low-substituted), maize starch, polyvinylpyrrolidone, copolymers of vinylpyrrolidone with other vinyl derivatives (copovidone), hydroxypropylcellulose, hydroxypropylmethylcellulose, microcrystalline cellulose or starch, magnesium stearate, sodium stearylfumarate, talc, hydroxypropylmethylcellulose, carboxymethylcellulose, cellulose acetate phthalate, polyvinyl acetate, water, water/ethanol, water/glycerol, water/sorbitol, water/polyethyleneglycol, propyleneglycol, cetylstearyl alcohol, carboxymethylcellulose or fatty substances such as hard fat or suitable mixtures thereof, into conventional galenic preparations such as plain or coated tablets, capsules, powders, suspensions or suppositories.

Tablets may be obtained for example by mixing the active substance or substances with one or more excipients and subsequently compressing them. The tablets may also consist of several layers. Examples of excipients are
inert diluents such as mannitol, sorbitol, xylitol, saccharose, calcium carbonate, calcium phosphate and lactose;
disintegrants such as croscarmellose sodium salt (cellulose carboxymethylether sodium salt, cross-linked), crospovidone, sodium starch glycolate, hydroxypropylcellulose (low-substituted) and maize starch;
binders such as polyvinylpyrrolidone, copolymers of vinylpyrrolidone with other vinyl derivatives (copovidone), hydroxypropylcellulose, hydroxypropylmethylcellulose, microcrystalline cellulose or starch;
lubricants such as magnesium stearate, sodium stearyl fumarate and talc;
agents for achieving delayed release such as hydroxypropylmethylcellulose, carboxymethylcellulose, cellulose acetate phthalate and polyvinyl acetate; and
pharmaceutically permitted colourings such as coloured iron oxides.

Blocking aldosterone reduces the mortality and morbidity in patients with pronounced weakness of the heart. If patients who are being treated with ACE inhibitors, angiotensin receptor antagonists, diuretics or beta-blockers for symptomatic weak heart and left ventricular failure suffer a myocardial infarct, it can be shown that additional treatment with the selective aldosterone blocker eplerenone improves their chances of survival and leads to a reduction in later hospital admissions (NEJM 348:1309-1321, 2003). The group of patients investigated also included diabetic patients who had left ventricular failure but no symptomatic weakness of the heart, as for this group of patients the risk of a cardiovascular event was similar to that of a patient with left ventricular failure and a symptomatic weakness of the heart. If this is taken into consideration, the discovery of the antidiabetic effect of the angiotensin II receptor blocker telmisartan disclosed here means that combining it with eplerenone in the after-treatment of a myocardial infarct is indicated particularly in patients who are already suffering from diabetes and in any case diabetic proteinuria or who are suspected of being pre-diabetic. The present invention therefore also relates to pharmaceutical formulations in which the two active substances telmisartan and eplerenone are combined, e.g. one equivalent of 40-320 mg, preferably 80 or 160 mg of telmisartan and one equivalent of 20-200 mg, preferably 25 or 50 mg of eplerenone. A preferred formulation consists of two-layer tablets. For treating patients showing symptoms of both dyslipidaemia (e.g. hypertriglyceridaemia or hypercholesterolaemia) the additional combination with a lipid lowering agents such as simvastatin or atorvastatin in the usual dosage of 2.5-40 mg, preferably 5, 10, 15, 20, 25, 30, 35 or 40 mg is frequently a good idea. A corresponding formulation of telmisartan, eplerenone and atorvastatin or simvastin may be prepared, for example, in the form of a three-layer tablet. The active substance eplerenone is used, in particular, in micronised form with a D₉₀ particle size of 25-400 microns (cf. WO 00/33847).

Mefformin is a tried and tested antidiabetic agent which achieves its main effect by lowering the excessive glucose production in the liver of a diabetic. In monitoring the treatment of diabetes mellitus the HbA1c value, the product of a non-enzymatic glycation of the haemoglobin B chain, is of exceptional importance. As its production depends essentially on the blood sugar level and the life of the erythrocytes, the HbA1c in the sense of a “blood sugar memory” reflects the average blood sugar levels of the preceding 4-6 weeks. Diabetic patients whose HbA1c value is consistently well adjusted by intensive diabetes treatment (i.e. <6.5 % of the total haemoglobin in the sample), are significantly better protected against diabetic microangiopathy. Metformin on its own achieves an average improvement in the HbA1c value in the diabetic of the order of 1.0-1.5%. This reduction of the HbA1C value is not sufficient in all diabetics to achieve the desired target range of <6.5% and preferably <6% HbA1c. Therefore, additional therapeutic measures are needed to increase the effect of mefformin.

Within the scope of the present invention it has surprisingly been found that telmisartan acts as an activator of PPAR-gamma. Thus, telmisartan has the potential to increase the insulin sensitivity of fat, muscle and liver tissue, and thereby lower the blood sugar. This makes telmisartan a particularly suitable combination partner for antidiabetics such as mefformin or repaglinide (promoting the release of insulin from the B-cells of the pancreas), as its effect is based on a different principle and thus favourably intensifies the effect of these active substances. Formulations of a combination of repaglinide and telmisartan contain, for example, one equivalent of 0.25 to 5 mg, preferably 0.25 to 2 mg, and most preferably 0.5 or 1 or 2 mg repaglinide and one equivalent of 40-320 mg, preferably 80 or 160 mg of telmisartan.

A combination of telmisartan and mefformin is particularly suitable in obese type 2 diabetics as on the one hand mefformin unlike other oral antidiabetics does not lead to an increase in body weight, and on the other hand telmisartan reduces insulin resistance as an important feature and cause of the raised blood sugar levels in these type 2 diabetics. In the majority of obese type 2 diabetics and also prediabetics an increase in blood pressure is detected which is also one of the criteria of metabolic syndrome. For this group of patients telmisartan is a preferred antihypertensive the additional properties of which as an insulin sensitizer interact favourably with a tried and tested antidiabetic such as mefformin, in order to treat different aspects of the diseases type 2 diabetes, type 2 prediabetes or metabolic syndrome or insulin resistance at the same time and in the same way. By the additional administration of telmisartan an additional improvement in the HbA1c value of the order of 0.25-2%, preferably 0.25-1% and most preferably 0.25-0.5% can be achieved. An improvement in the HbA1c value of less than 0.25% of the total haemoglobin constitutes a sensible contribution to the treatment of a type 2 diabetic but is currently not reliably capable of being measured. As the UKPDS (United Kingdom Prospective Diabetes Study) has shown, lowering raised blood pressure is just as effective as a treatment to lower blood sugar in reducing late complications in type 2 diabetics such as nephropathy, neuropathy, retinopathy and all macrovascular complications. Thus, the proposed combination of telmisartan and metformin constitutes a major contribution to the reduction or even prevention of the serious consequences of diabetes.

Formulations of a combination of mefformin and telmisartan contain, for example, on equivalent of 450-900 mg, preferably 500 or 850 mg metformin and one equivalent of 40-320 mg, preferably 80 or 160 mg of telmisartan. Metformin hydrochloride dissolves easily and can readily be formulated with excipients such as binders and lubricants. A preferred formulation consists of two-layer tablets. The combination of the preferred quantities of active substance and excipient results in the following compositions:

TABLE 1
Tablets containing 80 mg telmisartan

| | Telmisartan/Metformin | |
|---|---|---|
| | 80/500 mg | 80/850 mg |
| Metformin hydrochloride | 643 mg | 1094 mg |
| (corresponding to 500 mg and 850 mg metformin) | | |
| Excipients (binders and lubricants) at least | 27 mg | 46 mg |
| Telmisartan SD-granules | 135 mg | 135 mg |
| (corresponding to 80 mg telmisartan) | | |
| Excipients consisting of telmisartan tablet matrix | 345 mg | 345 mg |
| (sorbitol and lubricant) | | |
| Total Tablet (at least) | 1150 mg | 1620 mg |

TABLE 2
Tablets containing 160 mg of telmisartan

| | Telmisartan/Metformin | |
|---|---|---|
| | 160/500 mg | 160/850 mg |
| Metformin hydrochloride | 643 mg | 1094 mg |
| (corresponding to 500 mg and 850 mg | | |
| metformin) | | |
| Excipients (binders and lubricants) at least | 27 mg | 46 mg |
| Telmisartan SD-granules | 270 mg | 270 mg |
| (corresponding to 160 mg telmisartan) | | |
| Excipients consisting of telmisartan tablet | 690 mg | 690 mg |
| matrix (sorbitol and lubricant) | | |
| Total Tablet (at least) | 1630 mg | 2100 mg |

Formulations of a combination of repaglinide and telmisartan contain, for example, one equivalent of 0.25 to 5 mg, preferably 0.25 to 2 mg, and most preferably 0.5 or 1 or 2 mg of repaglinide and one equivalent of 40-320 mg, preferably 80 or 160 mg of telmisartan.

### EXAMPLES

### Example 1

### Telmisartan, Losartan and Irbesartan Do Not Bind In Vitro to the PPARgamma Ligand Binding Domain

Protein containing the human PPARgamma-ligand binding domain (LBD) is prepared as a GST fusion protein in E.coli and purified by affinity chromatography. To do this, a DNA section which codes for the amino acids 205-505 of the human PPARgamma2 transcription factor (cf. Genbank entry U79012) is subcloned via the additionally introduced restriction cutting sites BamH I and Xho I into the expression vector pGEX-4T-1 (Amersham) and the sequence of the section is monitored. The fusion protein is expressed in the E.coli strain BL21(DE3) recommended for pGEX vectors after induction with 0.2 mM IPTG for 4 hours at 25° C. The bacteria are pelleted after the induction and frozen in batches in PBS, pH 7.4. After opening up in a French Press, the dissolved GST-PPARgamma-LBD-fusion protein is purified using a GSTrap column (Pharmacia). Elution is carried out by the addition of 20 mM reduced glutathione.

The GST-PPARgamma-LBD-protein fractions are desalinated using a HiTrap desalting column (Pharmacia) and the protein concentration is determined using a standard assay.

Protein containing the human RXRalpha ligand binding domain (LBD) is prepared as a His tag fusion protein in E.coli and purified by affinity chromatography. To do this a DNA section which codes for the amino acids 220-461 of the human RXRalpha transcription factor (cf. Genbank entry NM_{—}002957, nt 729-1457) is subcloned via the additionally introduced restriction cutting sites BamH I and Not I into the expression vector pET28c (Novagen) and the sequence of the section is monitored. The fusion protein is expressed in the E.coli strain BL21(DE3) recommended for pET vectors after induction with 0.2 mM IPTG for 4 hours at 25° C. The bacteria are pelleted after the expression and frozen in batches in PBS, pH 7.4. After opening up in a French Press, the dissolved His-RXRalpha-LBD-fusion protein is purified using a HiTrap chelating column (Pharmacia). Elution is carried out using a 500 mM imidazole step. The His-RXRalpha-LBD protein fractions are desalinated using a HiTrap desalting column (Pharmacia) and the protein concentration is determined using a standard assay.

a) AlphaScreen

Alpha Screen assays were first described in Ullmann E F et al, Proc Natl Acad Sci USA (1994) 91:5426-5430. The measurements carried out within the scope of this Example were carried out as described by Glickman J F et al., J Biomol Screen (2002) 7:3-10. The assay buffer consists of 25 mM Hepes pH7.4, 100 mM NaCl,1 mM DTT, 0.1% Tween-20, 0.1% BSA. 3 nM GST-PPARgamma-LBD fusion protein, 15 nM biotinylated LXXLL peptide of the cofactor CBP (corresponding to the peptide disclosed on page 218 of Mukherjee R et al., J Steroid Biochem Mol Biol (2002) 81:217-225 with an additional N-terminal cysteine), and in each case 10 μg/ml of anti-GST-acceptor beads or streptavidine-donor beads (Applied Biosystems) are incubated in a total volume of 12.5 μl in the presence of different concentrations of a test substance (in DMSO) for 4 hours at ambient temperature. The final DMSO concentration in the assay is 1% (v/v). A 1% DMSO solution is used as the background control (NSB). The measurement is done using a Packard fusion measuring device.

| | |
|---|---|
| telmisartan | rosiglitazone |

| conc./M | MW | SD | MW | SD |
|---|---|---|---|---|
| NSB | 619 | 21 | 573 | 17 |
| 1.00E−08 | | | 820 | 18 |
| 3.00E−08 | 642 | 41 | 1720 | 48 |
| 1.00E−07 | 606 | 10 | 8704 | 59 |
| 3.00E−07 | 644 | 56 | 27176 | 1232 |
| 1.00E−06 | 677 | 14 | 43233 | 1083 |
| 3.00E−06 | 720 | 35 | 52691 | 3771 |
| 1.00E−05 | 847 | 82 | 56366 | 4303 |
| 5.00E−05 | 1111 | 135 | | |

Unlike rosiglitazone, a PPARgamma-agonist known from the literature with binding in the LBD, the use of increasing concentrations of telmisartan, losartan and irbesartan (concentrations of up to 50 μM) does not result in any direct activation of the PPARgamma-LBD and hence in any significant recruiting of the LXXLL peptide.

b) SPA Assay

A description of the SPA assay format can be found in Mukheriee R et al., J Steroid Biochem Mol Biol (2002) 81:217-225. The assay buffer consists of 20 mM Tris pH 7.5, 25 mM KCl, 10 mM DTT and 0.2% Triton X-100. 30 nM GST-PPARgamma-LBD fusion protein, 30 nM His-RXRalpha-LBD, anti-GST-antibody (1:600, Amersham Pharmacia), 0.25 mg protein A SPA PVT antibody-binding beads (Amersham Pharmacia), 30 nM ³H-labelled rosiglitazone are incubated with dilutions of the test substance for 5 hours at room temperature in a total volume of 100 μl.

10 μM of unlabelled rosiglitazone is added as background control (NSB) instead of the radioactive rosiglitazone, and the solvent used, e.g. DMSO, is added as the maximum value (Bmax) instead of a test substance.

After the incubation the test preparations are centrifuged for 5 minutes at 2000 rpm in a Hettich Universal 30 Rf centrifuge and measured using a Packard TopCount NXT.

| | | |
|---|---|---|
| telmisartan | irbesartan | losartan |

| conc/M | MW | SD | MW | SD | MW | SD |
|---|---|---|---|---|---|---|
| NSB | 217 | 9 | 217 | 9 | 217 | 9 |
| Bmax | 911 | 15 | 911 | 15 | 911 | 15 |
| 1.00E−07 | 837 | 49 | 913 | 54 | 915 | 43 |
| 3.00E−07 | 802 | 28 | 810 | 49 | 835 | 11 |
| 1.00E−06 | 818 | 27 | 815 | 51 | 901 | 10 |
| 3.00E−06 | 818 | 20 | 779 | 26 | 814 | 53 |
| 1.00E−05 | 703 | 30 | 723 | 37 | 787 | 46 |
| 3.00E−05 | 691 | 222 | 648 | 40 | 784 | 96 |
| 1.00E−04 | 545 | 18 | 510 | 81 | 611 | 17 |

In contrast to direct PPARgamma-agonists which bind to the PPARgamma-LBD, no concentration-dependent displacement of the radioactive rosiglitazone from the binding pocket takes place even in the presence of very large excesses of telmisartan, losartan or irbesartan.

c) NMR Investigations

In contrast to a direct PPARgamma ligand, e.g. rosiglitazone, no interaction of the test substance with amino acids in the binding pocket takes place during the measurement of the ¹⁵N TROSY spectrum of the PPARgamma-LBD in the presence of the test substance telmisartan. The amino acids of the binding pocket have the same position in the presence of the test substances as in the absence of a ligand.

### Example 2

### Preparation of a Stably Transformed PPARgamma Reporter Cell Line

A DNA section which codes for amino acids 205-505 of the human PPARgamma2 transcription factor (corresponding to nucleotides 703-1605 of Genbank sequence U79012) is incorporated into the Multiple Cloning Site of the vector pFA-CMV (Stratagene) via additionally introduced restriction cutting sites BamH I and Hind III and the sequence is verified. The resulting plasmid pFA-CMV/hPPARgamma2-LBD codes N-terminally of the PPARgamma-LBD in the same reading frame for a Gal4 DNA binding domain. In addition the plasmid codes for a neomycin resistance.

The cell line CHO-K1 (ATCC CCL-61) is cotransfected with the plasmids pFA-CMV/hPPARgamma2-LBD and pFR-Luc (Stratagene). pFR-Luc codes for the luciferase gene under the control of a five-times repeated yeast Gal4 binding site. The transfection is carried out with lipofectamine2000 in accordance with the manufacturer's instructions.

After transfection the cells are cultivated in medium (Ham's F12 with 10% foetal calf serum) in the presence of 0.5 mg/ml G-418. After six days' cultivation the cells are passaged and kept in culture for another 10 days. The resulting neomycin-resistant colonies are picked out under the microscope and transferred into 96 well-dishes and cultured. Various transformed cell lines are obtained with the plasmids contained therein (e.g. clone no.10, 11, 13 etc), which are kept in the culture medium.

The cell lines are examined for the inducibility of the luciferase gene using a PPARgamma agonist, e.g. rosiglitazone, and react with an increased luciferase signal to stimulation by the PPARgamma agonist.

### Example 3

### Telmisartan, Losartan and Irbesartan Activate PPARgamma at Cellular Level

The CHO-K1 cell line derived from the transformed clone 11 of Example 2 is seeded in 96-well flat-bottomed dishes in a density of 3×10⁴ cells/200 μl/well and cultivated overnight in Ham's F-12 medium with 10% foetal calf serum and 0.5 mg/ml G-418. After 24 hours the medium is changed for one without any added G-418.

The test substances are brought to 100 times the desired concentration with a suitable solvent, e.g. DMSO, and diluted 1:100 with the medium placed in the cell culture plate. The solvent used, e.g. DMSO, is used as the background control in the same concentration.

24 hours after the addition of the substance the supernatants are discarded and the cells are washed twice with 150μl washing buffer (25 mM Tricine, 16.3 mM MgSO₄, pH7.8). After the washing steps 50 μl of washing buffer with 150 μl of luciferase assay buffer (25 mM Tricine, 0.5 mM EDTA, 0.54 mM NaTPP, 16.3 mM MgSO₄, 1.2 mM ATP, 0.05 mM luciferine, 56.8 mM 2-mercaptoethanol, 0.1% Trition X-100, pH7.8) are added to each test preparation. Luminescence is measured after a five minute wait using a Packard TopCount NXT. The luciferase activity is obtained by integrating the relative luciferase units (RLU) of the first ten seconds after the start of measurement.

| | telmisartan | | irbesartan | | losartan | | rosiglitazone | |
|---|---|---|---|---|---|---|---|---|
| conc/M | MW | SD | MW | SD | MW | SD | MW | SD |
| NSB | 466 | 188 | 466 | 188 | 466 | 188 | 741 | 141 |
| 1.00E−08 | | | | | | | 2761 | 178 |
| 3.00E−08 | | | | | | | 8256 | 708 |
| 1.00E−07 | | | | | | | 35265 | 2947 |
| 3.00E−07 | 760 | 255 | 491 | 70 | 874 | 475 | 86859 | 6139 |
| 1.00E−06 | 2859 | 455 | 657 | 65 | 589 | 70 | 106252 | 30018 |
| 3.00E−06 | 24498 | 2290 | 1028 | 342 | 672 | 88 | 143232 | 14064 |
| 1.00E−05 | 61397 | 7853 | 3292 | 556 | 709 | 163 | 150989 | 24245 |
| 3.00E−05 | 58790 | 2055 | 22133 | 4202 | 3271 | 585 | | |
| 1.00E−04 | | | 29600 | 6936 | 11322 | 1668 | | |

The angiotensin II receptor antagonist telmisartan brings about a particularly potent activation of the PPARgamma pathway in the PPARgamma reporter cell line. Activation by other angiotensin II receptor antagonists such as losartan and irbesartan takes place only at higher test concentrations and to a lesser extent.

### Example 4

### Experiments with 3T3-L1 Adipocytes and PC12W Cells

3T3-L1 mouse preadipocytes are cultivated in DMEM (Dulbecco's modified eagle medium) with 10% foetal calf serum (FBS). PC12W cells are cultivated in DMEM with 5% FBS and 10% equine serum. In both cases the media contain 1% penicillin/streptomycin.

The differentiation of adipocytes is induced 2-3 days after cell confluence by adding a differentiating solution. This contains
1 μmol/L of dexamethasone,
0.5 mmol/L of 3-isobutyl-1-methylxanthine,
1.67 μmol/L of insulin, and
10% FBS.

For comparison, differentiation is also induced with a differentiating solution which additionally contains telmisartan. After 48 hours (day 2) the medium is replaced by DMEM containing 10% FBS and 1.67 μmol/L of insulin or 10% FBS and 1.67 μmol/L insulin and telmisartan. Then the cells are stimulated for another 48 hours before finally being analysed (day 4).

Lipid Accumulation in 3T3-L1 Adipocytes

Cells are washed with PBS and fixed with a 3.7% formaldehyde solution for 2 minutes. After fixing, the cells are stained for 1 hour at ambient temperature with a 0.5% stock solution of Oil Red-O in isopropanol diluted 3:2 with water. After washing, the cells are examined under a light microscope.

10 μmol/L of telmisartan bring about an increased accumulation of lipids which is made visible by increased staining with Oil Red-O. The differentiation of 3T3-L1 adipocytes is also promoted by telmisartan.

Stimulation of the aP2 Expression in 3T3-L1 Cells

RNA isolation, reverse transcription and quantification of gene expression are carried out using an ABI 7000 sequence detection system-for real time PCR (described in Janke et al, Diabetes 51:1699-707, 2002). The endogenous control used for the real time PCR consists of the household genes 18S rRNA and hypoxanthine guanine phosphoribosyl transferase (hprt).

The induction observed is dependent on the concentration of telmisartan used. 10 μmol/L of telmisartan stimulate the expression of the adipogenic marker gene Adipose Protein 2 (aP2) in 3T3-L1 cells by a factor of 3.1±0.3 (p<0.01). By comparison, a concentration of 10 μmol/L of the PPARgamma ligand pioglitazone stimulates aP2 expression by a factor of 4.5±1 (p<0.01).

Transcription Reporter Assays

In order to investigate whether the induction of the adipogenesis by telmisartan is the result of stimulation of the PPARgamma activity, transfection experiments are carried out with PPRE (PPAR Response Element) Reporter constructs. The transient transfection and the luciferase assays used are described in Kintscher et al, Circ Res. 91 :e3544, 2002. 3T3-L1 adipocytes (day 4) or PC12W cells are transfected with Lipofectamine 2000 (Invitrogen) in the presence of 1 μg (for 3T3-L1 cells) or 50 ng (for PC12W cells) of a reporter construct, PPARgamma2 and RXRalpha expression vectors and 10 ng of a Renilla Luciferase Reporter control vector. The reporter construct is a fusion of 3xAcyl-CoA oxidase PPAR Response Element (PPRE) with Tk-luciferase. The PPARgamma2 and RXRalpha expression vectors used correspond to the vectors described by Elbrecht et al, Biochem Biophys Res Corn 224: 431-437, 1996 and Joseph et al, J Biol Chem 277(13): 11019-11025, 2002. The Luciferase Reporter control vector is the plasmid pRL-CMV (Promega). After 4 hours the transfection medium is replaced by DMEM with 10% FBS which additionally contains telmisartan, pioglitazone or the carrier DMSO. Luciferase activity is measured after 24 hours.

Treatment of 3T3-L1 adipocytes with 10 μmol/L telmisartan leads to the induction of the transcriptional activity of PPARgamma by a factor of 3.4±0.9 (p<0.05) compared with induction by a factor of 5.2±1.1 by 10 μmol/L pioglitazone.

PC12W cells are AT₁-receptor-deficient. PPARgamma 2 and its heterodimeric partner RXRalpha are overexpressed in PC12W cells and the PPARgamma-dependent transcription is measured in the presence and absence of 10 μmol/L telmisartan or pioglitazone. As PC12W cells do not express PPARgamma, no regulation of the PPARgamma activity is measured in the absence of exogenous PPARgamma2/RXRalpha. After overexpression of the PPARgamma 2/RXRalpha heterodimer, however, telmisartan also induces the PPARgamma activity by a factor of 1.9±0.4 (p<0.05) in the AT₁-receptor-deficient PC 2W cells. By comparison, pioglitazone induces PPARgamma activity by a factor of 4.2±1.4 (p<0.01). This demonstrates that the activation of the PPARgamma activity by telmisartan takes place independently of the blocking of the AT₁-receptor.

The data also show that telmisartan concentrations which are necessary in order to stimulate the PPARgamma activity can be achieved in the blood plasma of patients being treated with telmisartan for high blood pressure. This means that high blood pressure treatment with telmisartan is also additionally able to improve insulin sensitivity, which has a positive effect on the blood sugar level.

### Example 5

### Examples of Formulations

Tablet 1

Tablets having the following composition are obtained by direct compression of the telmisartan sodium salt with excipients and magnesium stearate:

| Ingredients: | mg |
|---|---|
| telmisartan sodium salt | 41.708 |
| mannitol | 149.542 |
| microcrystalline cellulose | 50.000 |
| croscarmellose sodium salt | 5.000 |
| magnesium stearate | 3.750 |
| total | 250.000 |

Tablet 2

Tablets having the following composition are obtained by direct compression of the telmisartan sodium salt with excipients and magnesium stearate:

| Ingredients: | mg |
|---|---|
| telmisartan sodium salt | 83.417 |
| sorbitol | 384.083 |
| polyvidone K25 | 25.000 |
| magnesium stearate | 7.500 |
| total | 500.000 |

Tablet 3

Hydrochlorothiazide, telmisartan sodium salt, sorbitol and red iron oxide are mixed in a free fall blender, passed through a 0.8 mm screen and, after the addition of magnesium stearate, processed in a free fall blender to obtain a powdered mixture.

This combination of active substances and excipients is than compressed with a suitable tablet press (e.g. Korsch EKO or Fette P1200) to form tablets. Tablets with the following composition are obtained, the quantity of telmisartan sodium salt contained in each tablet corresponding to a quantity of 80 mg of the free acid of telmisartan.

| Ingredient | mg/tablet | % |
|---|---|---|
| telmisartan sodium salt | 83.417 | 13.903 |
| hydrochlorothiazide | 12.500 | 2.083 |
| sorbitol | 494.483 | 82.414 |
| red iron oxide | 0.600 | 0.100 |
| magnesium stearate | 9.000 | 1.500 |
| total | 600.000 | 100.000 |

The telmisartan sodium salts of the tablets of the three batches dissolves in 900 ml of 0.1 M phosphate buffer, pH 7.5, at a rate of 92±1.5%, 96±1.8% and 100±1.0%, respectively, after 30 minutes stirring (75 rpm). The hydrochlorothiazide dissolves in 900 ml of 0.1 M HCl (100 rpm) after 30 minutes at a rate of 69±6.3%, 72±2.1% and 78±1.8%, respectively.

1. A method of treating people in whom type 2 diabetes mellitus has been diagnosed or who are suspected of prediabetes, or for treating metabolic syndrome and insulin resistance in patients with normal blood pressure said method comprising administering a pharmaceutical composition comprising a pharmaceutically effective amount of an angiotensin II receptor antagonist or a salt thereof.

2. The method according to claim 1, wherein for the subjects to be treated the fasting blood sugar level exceeds 125 mg glucose per dl of plasma.

3. The method according to claim 1, wherein for the subjects to be treated the fasting blood sugar level is 110-125 mg glucose per dl of plasma.

4. The method according to claim 1, wherein for the subjects to be treated a blood sugar level of more than 200 mg of glucose per dl of plasma is measured 2 hours after taking 75 g of glucose on an empty stomach.

5. The method according to claim 1, wherein for the subjects to be treated a blood sugar level of 140-200 mg of glucose per dl of plasma is measured 2 hours after taking 75 g of glucose on an empty stomach.

6. The method according to claim 1, wherein for the subjects to be treated the blood level for triglycerides exceeds 150 mg/dl.

7. The method according to claim 6, wherein for the subjects to be treated the blood level for HDL is less than 40 mg per dl of plasma in women and less than 50 mg per dl of plasma in men.

8. The method according to claim 7, wherein for the subjects to be treated the fasting blood sugar level exceeds 110 mg glucose per dl of plasma.

9. The method according to any one of claims 1, 2 or 4, wherein for the subjects to be treated the systolic blood pressure exceeds a value of 140 mm Hg and the diastolic blood pressure exceeds a value of 90 mm Hg.

10. The method according to any one of claims 1, 2 or 4, wherein for the subjects, to be treated the systolic blood pressure exceeds a value of 130 mm Hg and the diastolic blood pressure exceeds a value of 80 mm Hg.

11. The method according to claim 10, wherein for the subjects to be treated the ratio of waist measurement to hip measurement in women exceeds a value of 0.8 in women and a value of 1 in men.

12. The method according to claim 1, wherein the angiotensin II receptor antagonist has the property of activating the expression of a stably transfected luciferase gene after the addition of a stably transformed PPARgamma reporter cell line to the culture medium, without binding in vitro to the PPARgamma ligand binding domain.

13. The method according to claim 12, wherein
the angiotensin II receptor antagonist does not exhibit any binding in vitro to the ligand binding domain of a human PPARgamma receptor while
the angiotensin II receptor antagonist leads to the induction of a luciferase activity when it is added to the culture medium of a stably transformed cell line which
expresses a fusion protein consisting of the ligand binding domain of the human PPARgamma transcription factor and the yeast GAL4 DNA binding domain and
contains a luciferase gene under the control of a five-times repeated yeast Gal4 binding site.

14. The method according to claim 1, wherein the angiotensin II receptor antagonist is the active substance telmisartan.

15. The method according to claim 1, wherein the formulation of the pharmaceutical composition contains 20-200 mg telmisartan.

16. The method according to claim 1, wherein the angiotensin II receptor antagonist is combined with a diuretic.

17. The method according to claim 16, wherein the formulation of the pharmaceutical composition contains 10-50 mg of HCTZ or chlorthalidone.

18. A Pharmaceutical composition containing a pharmaceutically effective amount of telmisartan in conjunction with a pharmaceutically effective amount of
a) amlodipine or nifedipine,
b) eplerenone or spironolactone,
c) simvastatin or atorvastatin,
d) rosiglitazone or pioglitazone or repaglinide or mefformin,
e) dipyridamole or clopidogrel, optionally combined with acetylsalicylic acid, a sulphonylurea,
f) an aldosterone antagonist,
g) an HMG-Co A reductase inhibitor,
h) a DPP4 inhibitor,
i) a sulphonylurea or
j) a thrombocyte aggregation inhibitor.
